(19) United States
(12) Plant Patent Application Publication
(10) Pub. No.: US 2013/0347151 P1
(43) Pub. Date: Dec. 26, 2013

(54) Blueberry plant named 'Cipria'

(50) ;

(71) Applicants: Jessica Scalzo, Havelock North (NZ); Narandra Patel, Hamilton (NZ)

(72) Inventors: Jessica Scalzo, Havelock North (NZ); Narandra Patel, Hamilton (NZ)

(21) Appl. No.: 13/815,549

(22) Filed: Mar. 8, 2013

Related U.S. Application Data
(60) Provisional application No. 61/664,155, filed on Jun. 26, 2012.

(51) Int. Cl. A01H 5/00

(52) U.S. Cl. PLT/157

(57) ABSTRACT

A new and distinct blueberry variety is described. The variety results from selection among a population of seedlings derived from the deliberate open pollination of the variety ‘Summit’ (not patented). The new variety is distinguished from others by the fruit harvest season, fruit size, flavour and bloom, along with the plant habit. Fruit of ‘Cipria’ is medium to large in size with a strong bloom; the mature plant has a semi-upright to spreading habit.

### CROSS REFERENCE TO RELATED APPLICATION

This application claims the benefit of U.S. Provisional Application No. 61/664,155 filed Jun. 26, 2012, the entire disclosure of which is incorporated by reference in its entirety.

### GENUS AND SPECIES OF PLANT CLAIMED

Vaccinium corymbosum

### VARIETY DENOMINATION

‘Cipria’

### BACKGROUND TO THE INVENTION

A plant variety named ‘RH48’ was selected from among a population of seedlings derived from the open pollination of the variety ‘Summit’ (not patented), located at Ruakura, Hamilton, New Zealand. Along with a number of other selections ‘RH48’ was identified as having potential for test and evaluation and was shipped to Gilten, Germany in 2001. In 2002 it was identified as having potential as a new variety and was asexually propagated. The resulting plants were planted out at Gilten and were subsequently found to be true to type demonstrating that the characteristics of the new variety are stable and transmitted without change through succeeding generations. Propagation has since been carried out through both soft and hard wood cuttings. ‘RH48’ was later designated ‘Cipria’.

### SUMMARY OF THE INVENTION

‘Cipria’ is characterised by its early to mid season harvest maturity, medium to large fruit size, small pedicel scar, strong bloom, and semi-upright to spreading habit. ‘Cipria’ is distinguished from a number of other varieties by the following characteristics:

‘Cipria’ has a semi-upright to spreading habit while ‘Island Blue’ (not patented) and ‘Mondo’ (not patented) have an upright habit.

‘Cipria’ produces oblate medium to large fruit, averaging 2.2 g, with a strong bloom, whereas ‘Nui’ (U.S. Plant Pat. No. 6,699) produces flat large fruit, averaging 3 g, with medium boom and lower soluble solid content, under the same conditions in Germany.

### BRIEF DESCRIPTION OF THE DRAWINGS

FIG. 1 shows mature, seven year old, ‘Cipria’ plants with fruit.

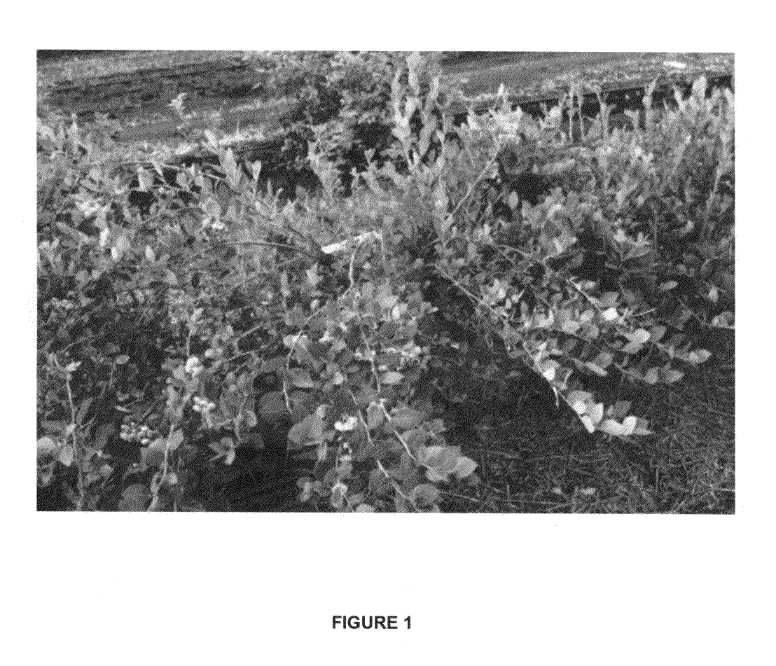

FIG. 2 shows fruit of ‘Cipria’ on the plant.

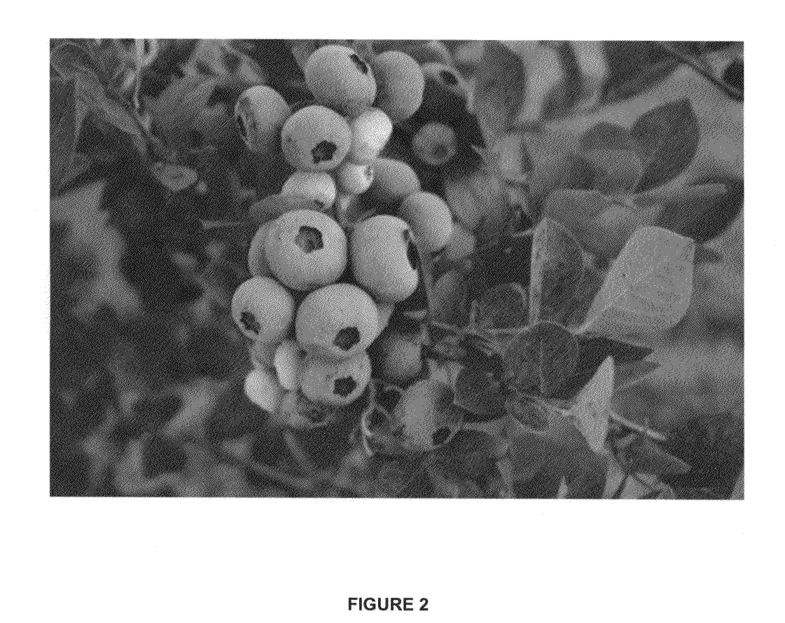

### DETAILED DESCRIPTION

The following is a description of the new variety with color terminology in accordance with The Royal Horticultural Society Colour Charts (R.H.S.C.C.) 2001 edition.

Observations were made on specimens grown at Gilten, Germany and at Ruakura, Hamilton, New Zealand. The observations were made in the 2010-2012 seasons on 7-9 year old bushes.
Plant and foliage:
   Plant.—medium size at maturity, managed under standard orchard practice. Growth habit is semi-upright to spreading and fruit are borne only on one-year-old shoots.
   Leaves.—mature fully expanded leaves are medium to short at an average of approximately 52.0 mm long and 32.4 mm wide. The pinnate leaves are oblate in shape with smooth margins. The leaves are coloured near green 137A with very low and uniform glossiness on the upper surface and venation coloured near red purple 60A on the underside. The petioles are an average of approximately 3.5 mm long.
Inflorescence: Flowers were an average of approximately 7.3 mm long and 7.2 mm wide with very weak anthocyanin in the petals which are coloured near white 155C.
Fruit:
   Immature fruit.—coloured near yellow green 144C to 144D.
   Mature fruit.—medium to large in size, with an average fresh weight of 2.2 g and diameter of 17.2 mm. The fruit have a medium to soft firmness under New Zealand conditions and medium firmness in Germany—influenced by environmental conditions. The fruit have a strong bloom with medium blue skin colour after the bloom is removed. The fruit are generally oblate in shape with a small dry scar and an average brix of 12.2%.
   Seeds.—there is an average of between 18 to 32 seeds per fruit with an average length of 1.8 mm, coloured near greyed-orange 175A.
Events: The timing of the beginning of flowering is early compared to standard V. corymbosum varieties, while the timing of the beginning of fruit ripening is early to medium. Under New Zealand conditions 50% of flowering occurs late August and 50% of fruit ripening occurs mid December.
Pest and disease: ‘Cipria’ does not appear particularly resistant or susceptible to any particular pests or diseases known to afflict V. corymbosum.
Hardiness: The plant cold hardiness according to the American zone classification has not been determined. ‘Cipria’ has a chilling requirement of between 600-800 hours.

1. A new and distinct blueberry plant substantially as illustrated and described herein.
